(19) United States
(12) Patent Application Publication
(10) Pub. No.: US 2024/0368053 A1
(43) Pub. Date: Nov. 7, 2024

(54) USE OF A CATALYST SYSTEM IN THE PRODUCTION OF 1,3-BUTADIENE FROM ETHANOL IN TWO STAGES

(71) Applicant: SYNTHOS S.A., OSWIECIM (PL)

(72) Inventors: Ewelina JANKOWIAK, OSWIECIM (PL); Szymon SKOWRONEK, OSWIECIM (PL); Magdalena SITKO, OSWIECIM (PL)

(21) Appl. No.: 18/552,756

(22) PCT Filed: Mar. 31, 2022
(86) PCT No.: PCT/EP2022/058736

§ 371 Date: Sep. 27, 2023

(30) Foreign Application Priority Data
Apr. 1, 2021 (EP) 21461532.0

(51) Int. Cl. C07C 1/207; B01J 21/08; B01J 23/20; B01J 35/00; B01J 37/02; B01J 37/08; C07C 45/51

(52) U.S. Cl. CPC C07C 1/2074; B01J 23/20; B01J 35/19; B01J 37/0236; B01J 37/08; C07C 45/512; B01J 21/08; C07C 2523/20

(57) ABSTRACT

The invention relates to a process for the production of 1,3-butadiene from ethanol, the process comprising a first stage and a second stage. Furthermore, the invention relates to a catalyst system for use in the production of 1,3-butadiene from ethanol. Moreover, the invention relates to the use of the catalyst system for the production of 1,3-butadiene from a feed comprising ethanol, and a plant comprising the catalyst system.

The present invention relates to a process for the production of 1,3-butadiene from ethanol, the process comprising a first stage and a second stage. Furthermore, the invention relates to a catalyst system for use in the production of 1,3-butadiene from ethanol. Moreover, the invention relates to the use of the catalyst system for the production of 1,3-butadiene from a feed comprising ethanol, and a plant comprising the catalyst system.

1,3-Butadiene is one of the most important raw materials in the synthetic rubber industry, where it is used as a monomer in the production of a wide range of synthetic polymers, such as polybutadiene rubbers, acrylonitrile-butadiene-styrene polymers, styrene-butadiene rubbers, nitrile-butadiene rubbers, and styrene-butadiene latexes. 1,3-Butadiene is, for example, obtained as a by-product of ethylene manufacturing in naphtha steam cracking and can be isolated by extractive distillation (Chem. Soc. Rev., 2014, 43, 7917; ChemSusChem, 2013, 6, 1595; Chem. Central J., 2014, 8, 53).

The depletion of non-renewable, fossil fuels-derived resources as well as environmental considerations have recently become strong driving forces for the exploration of renewable sources of 1,3-butadiene and its precursors. Of the wide range of the available renewable sources, biomass seems to have the greatest potential in the context of use for the production of 1,3-butadiene. This strategy has two main advantages: Independence from fossil fuels and reduction of CO₂ emissions (ChemSusChem, 2013, 6, 1595).

The conversion of ethanol, obtainable e.g. from biomass, to 1,3-butadiene may be performed in two ways reported in the literature: as one-step process (Lebedev process) and as two-step process (Ostromislensky process).

The one-step process, reported by Lebedev in the early part of the 20^{th} century, is carried out by direct conversion of ethanol to 1,3-butadiene, using multifunctional catalysts tuned with acid-base properties (J. Gen. Chem., 1933, 3, 698; Chem. Ztg., 1936, 60, 313).

On the other hand, the so-called two-step process may be performed by converting, in a first step, ethanol to acetaldehyde. The aim of this first step is to feed a second step or reactor with such mixture of ethanol and acetaldehyde. In the second step, conversion of the mixture to 1,3-butadiene over, for example, a silica-supported tantalum oxide catalyst takes place (Catal. Today, 2016, 259, 446). Tantalum oxide supported on silica is, however, inactive in the oxidation of ethanol to acetaldehyde. Therefore, and in order to move from a two-step to a one-step process, it is necessary to dope the 1,3-butadiene-generating catalyst with compounds that are active in the dehydrogenation of ethanol to acetaldehyde. Commonly used compounds active in this reaction are supported noble metals like silver or gold. WO 2012/015340 A1 teaches about 81-82% yield of 1,3-butadiene using a zirconia-silica catalyst doped with gold or gold with ceria. The feed contained 9% of acetaldehyde and the reaction was carried out at a weight hourly space velocity (WHSV) of 0.3 h⁻¹.

G. Pomalaza et al. disclose the direct conversion of ethanol to 1,3-butadiene with a catalyst comprising Zn(II) and Ta(V), supported on TUD-1, a sponge-like mesoporous silica with an irregular three-dimensional pore system (Green Chem., 2018, 20, 3203; Green Chem., 2020, 22, 2558). A stable selectivity of 68% towards 1,3-butadiene was achieved with Zn_{3.1}%—Ta_{1.9}%-TUD-1 at 350° C. and a WHSV of 5.3 h⁻¹. The synthesis of the catalysts comprising Zn(II) and Ta(V) involves the gelation by TEAOH of TEOS dissolved in ethanol with metal precursors complexed by tetraethylene glycol to ensure their dispersion. The resulting gel is dried and autoclaved, which creates the mesoporous morphology using tetraethylene glycol as a structure-directing agent. The resulting solid is calcined, ground in a mortar, and sieved to 125 μm, affording a white powder. Because the obtained materials are in the form of a powder, they would have to be shaped and formed to beads, pellets, tablets, etc. for any commercial, large-scale application. The production of 1,3-butadiene was carried out at a very small scale with only 30 mg of catalyst, which ensures good results since any potentially problematic phenomena related to heat and mass transfer can be neglected at this scale. The section “Conclusions” in Green Chem., 2018, 20, 3203 further discloses that the regeneration under air to remove deposed carbonaceous species was only partially successful, i.e. the described catalysts are not commercially applicable since the possibility to regenerate a catalyst is essential for running a commercial, large-scale plant. Moreover, the ethanol gas concentration in the feed was only 4.5 vol.-%. Such low ethanol feed concentration leads to a low content of heavy hydrocarbon side-products and thus the selectivity towards 1,3-butadiene is increased, however, a concentration of ethanol in the feed of only 4.5 vol.-% is not sufficient on a commercial scale.

US 2018/0208522 A1 relates to a catalyst comprising at least the element tantalum, and at least one mesoporous oxide matrix that has undergone an acid wash comprising at least 90% by weight of silica before washing, the mass of the element tantalum being in the range 0.1% to 30% of the mass of said mesoporous oxide matrix. Optionally, the catalyst comprises at least one element selected from the group consisting of groups 11 and 12 of the periodic table, the mass of said element being in the range 0.5% to 10% of the mass of said mesoporous oxide matrix. It describes that the inclusion of a group 12 element, particularly of zinc, enables the use of the catalyst in a one step process. The teaching of US 2018/0208522 A1 relies on acid washing of the mesoporous oxide support for increasing the selectivity towards 1,3-butadiene.

Thus, there is an ongoing need for providing straightforward and versatile processes for the production of 1,3-butadiene with high selectivity to and yield of 1,3-butadiene.

### SUMMARY OF THE INVENTION

According to the present invention, it was surprisingly found that a first stage contacting of a feed comprising ethanol with a first stage catalyst that catalyses the one-step process (conversion of ethanol to both acetaldehyde and 1,3-butadiene), followed by contacting of at least parts of the effluent of the first stage feed with a second stage catalyst that catalyses the second step of the two-step process (conversion of a mixture of ethanol and acetaldehyde to 1,3-butadiene), provides a straightforward and versatile process for the production of 1,3-butadiene from a feed comprising ethanol, with high selectivity to and yield of 1,3-butadiene.

Thus, in a first aspect, the present invention relates to a process for the production of 1,3-butadiene, the process comprising
i) a first stage contacting of a first stage feed comprising ethanol with a first stage catalyst, wherein the first stage catalyst comprises element MA1 and element MB1, MA1 is selected from the group consisting of zinc, copper, silver, gold, chromium, cerium, magnesium, platinum, palladium, cadmium, iron, manganese, ruthenium, cobalt, and nickel, and MB1 is selected from the group consisting of tantalum, zirconium, niobium, hafnium, titanium, and tin, to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde, wherein the second stage catalyst comprises element MB2, and MB2 is selected from the group consisting of tantalum, zirconium, niobium, hafnium, titanium, and tin, to produce a second stage effluent comprising 1,3-butadiene.

In a second aspect, the present invention relates to a catalyst system for use in the production of 1,3-butadiene from ethanol comprising i) a first stage catalyst comprising element MA1 and element MB1, wherein MA1 is selected from the group consisting of zinc, copper, silver, gold, chromium, cerium, magnesium, platinum, palladium, cadmium, iron, manganese, ruthenium, cobalt, and nickel, and MB1 is selected from the group consisting of tantalum, zirconium, niobium, hafnium, titanium, and tin, and ii) a second stage catalyst comprising element MB2, wherein MB2 is selected from the group consisting of tantalum, zirconium, niobium, hafnium, titanium, and tin.

Moreover, in a third aspect, the invention relates to the use of a catalyst system as defined herein for the production of 1,3-butadiene from a feed comprising ethanol, preferably to decrease the required amount of acetaldehyde in the first stage feed or to dispense altogether with acetaldehyde in the first stage feed.

Finally, and in a fourth aspect, the invention relates to a plant comprising the catalyst system as defined herein.

### DETAILED DESCRIPTION OF THE INVENTION

### 1) Process for the Production of 1,3-Butadiene

The process for the production of 1,3-butadiene of the present invention comprises the following stages
i) a first stage contacting of a first stage feed comprising ethanol with a first stage catalyst,
   wherein the first stage catalyst comprises element MA1 and element MB1,
      MA1 is selected from the group consisting of zinc, copper, silver, gold, chromium, cerium, magnesium, platinum, palladium, cadmium, iron, manganese, ruthenium, cobalt, and nickel, and
      MB1 is selected from the group consisting of tantalum, zirconium, niobium, hafnium, titanium, and tin,
   to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises element MB2, and
      MB2 is selected from the group consisting of tantalum, zirconium, niobium, hafnium, titanium, and tin,
   to produce a second stage effluent comprising 1,3-butadiene.

Due to the presence of MA1 and MB1, the first stage catalyst as defined herein catalyses both the conversion of ethanol to acetaldehyde and the conversion of the mixture of ethanol and acetaldehyde to 1,3-butadiene. It is thus a catalyst that may otherwise be used in the one-step (Lebedev) process, i.e. for the direct conversion of ethanol to 1,3-butadiene. The process according to the invention thus is particularly advantageous, because it enables the production of both acetaldehyde and 1,3-butadiene already in the first stage.

Preferably, the first stage effluent comprises ethanol, acetaldehyde and 1,3-butadiene.

According to one embodiment, the first stage feed only comprises ethanol (as the only 1,3-butadiene precursor) and no acetaldehyde.

According to another embodiment, the first stage feed comprises both ethanol and acetaldehyde.

The second stage catalyst as defined herein catalyses the conversion of a mixture of ethanol and acetaldehyde to 1,3-butadiene. Hence, it is a catalyst that may otherwise be used in the second part of the two-step (Ostromislensky) process. Contacting at least parts of the effluent of the first stage with the second stage catalyst is particularly advantageous, because it increases the yield of 1,3-butadiene inter alia by conversion of acetaldehyde produced in the first stage and by conversion of ethanol that did not react in the first stage to 1,3-butadiene.

According to one embodiment, element MB1 of the first stage catalyst is the same as element MB2 of the second stage catalyst.

According to another embodiment, element MB1 of the first stage catalyst is different from element MB2 of the second stage catalyst.

Preferably, the first stage contacting, or the second stage contacting, or both the first and the second stage contacting take place in a continuous flow fixed bed reactor.

Typically, the first stage effluent comprises ethanol, acetaldehyde and 1,3-butadiene.

According to another embodiment, the entirety of the first stage effluent is fed into the second stage, i.e. the second stage feed comprises the entirety of the first stage effluent. Preferably, the first stage effluent is the second stage feed.

Preferably, certain fractions of the first stage effluent are removed from the first stage effluent before it is fed into the second stage, so that the composition of the first stage effluent is changed before it is fed into the second stage.

The fractions that are separated from the first stage effluent, if applicable, may be
sent to work-up (e.g. as a separated 1,3-butadiene fraction),
recycled directly into the first stage feed, the second stage feed, or both the first and second stage feed (e.g. as a separated ethanol, or acetaldehyde, or ethanol and acetaldehyde fraction), or
purified and then recycled into the first stage feed, the second stage feed, or both the first and second stage feed (e.g. as a separated ethanol, or acetaldehyde, or ethanol and acetaldehyde fraction).

Preferably, the first stage effluent is separated into a first fraction comprising 1,3-butadiene, a second fraction comprising acetaldehyde, and a third fraction comprising ethanol, preferably wherein at least part of the second and the third fraction is recycled into the first stage feed, the second stage feed, or both the first and second stage feed. More preferably, the entirety of the second and the third fraction is recycled into the first stage feed, the second stage feed, or both the first and second stage feed.

Typically, the second stage effluent comprises ethanol, acetaldehyde and 1,3-butadiene.

According to one embodiment, the second stage effluent is sent to work-up in its entirety.

According to another embodiment, certain fractions of the second stage effluent are removed from the second stage effluent before it is sent to work-up, so that the composition of the second stage effluent is changed before it is sent to work-up.

The fractions that are separated from the second stage effluent, if applicable, may be
recycled directly into the first stage feed, the second stage feed, or both the first and second stage feed (e.g. as a separated ethanol, or acetaldehyde, or ethanol and acetaldehyde fraction), or
purified and then recycled into the first stage feed, the second stage feed, or both the first and second stage feed (e.g. as a separated ethanol, or acetaldehyde, or ethanol and acetaldehyde fraction).

Preferably, the second stage effluent is separated into a first fraction comprising 1,3-butadiene, a second fraction comprising acetaldehyde, and a third fraction comprising ethanol, preferably wherein at least part of the second and the third fraction is recycled into the first stage feed, the second stage feed, or both the first and second stage feed. More preferably, the entirety of the second and the third fraction is recycled into the first stage feed, the second stage feed, or both the first and second stage feed.

Thus, according to a preferred embodiment, there is at least one additional feed to the first stage, the second stage, or both the first and the second stage, that enables the feeding of recycled fractions of the first stage effluent, or of the second stage effluent, or of both effluents into the first stage, the second stage, or both the first and the second stage.

In one embodiment, the first stage feed is a mixture of fresh ethanol (i.e. ethanol that has not yet been used in the process according to the invention) and an additional feed comprising recycled ethanol.

In another embodiment, the first stage feed is a mixture of fresh ethanol and an additional feed, the additional feed comprising recycled ethanol and recycled acetaldehyde.

In another embodiment, the first stage feed is a mixture of fresh ethanol and fresh acetaldehyde (i.e. ethanol and acetaldehyde that have not yet been used in the process according to the invention, respectively), and an additional feed, the additional feed comprising recycled ethanol.

In another embodiment, the first stage feed is a mixture of fresh ethanol and fresh acetaldehyde and an additional feed, the additional feed comprising recycled ethanol and recycled acetaldehyde.

According to another preferred embodiment, there is an additional feed comprising fresh ethanol, or fresh acetaldehyde, or both fresh ethanol and fresh acetaldehyde, besides (at least part of) the first stage effluent being fed to the second stage.

In one embodiment, the second stage feed is a mixture of additional feed comprising fresh ethanol and (at least part of) the first stage effluent.

In another embodiment, the second stage feed is a mixture of additional feed comprising fresh ethanol and fresh acetaldehyde and (at least part of) the first stage effluent.

According to another preferred embodiment, the second stage feed is a mixture comprising recycled ethanol, or recycled acetaldehyde, or both recycled ethanol and acetaldehyde, and (at least part of) the first stage effluent.

In one embodiment, the second stage feed is a mixture of recycled ethanol and (at least part of) the first stage effluent.

In another embodiment, the second stage feed is a mixture of recycled ethanol and recycled acetaldehyde and (at least part of) the first stage effluent.

Preferably, the first stage contacting takes place at a weight hourly space velocity of from 0.2 to 10 h⁻¹, more preferably from 0.5 to 7 h⁻¹, most preferably from 0.5 to 5 h⁻¹.

Preferably, the second stage contacting takes place at a weight hourly space velocity of from 0.2 to 10 h⁻¹, more preferably from 0.5 to 7 h⁻¹, most preferably from 0.5 to 5 h⁻¹.

Most preferably, both the first and the second stage contacting take place at a weight hourly space velocity of from 0.2 to 10 h⁻¹, preferably from 0.5 to 7 h⁻¹, more preferably from 0.5 to 5 h⁻¹.

Preferably, the first stage contacting takes place at a pressure of 0 to 10 barg, preferably from 1 to 5 barg, more preferably from 1 to 3 barg.

Preferably, the second stage contacting takes place at a pressure of 0 to 10 barg, preferably from 1 to 5 barg, more preferably from 1 to 3 barg.

Preferably, both the first and the second stage contacting take place at a pressure of 0 to 10 barg, preferably from 1 to 5 barg, more preferably from 1 to 3 barg.

Preferably, the temperature of the first stage feed at the inlet to the first stage contacting is in a range of from 200 to 400° C., preferably from 325 to 375° C.

Preferably, the second stage contacting of the process for the production of 1,3-butadiene of the present invention takes place under adiabatic conditions, i.e. the heat required for the conversion of the mixture of ethanol and acetaldehyde to 1,3-butadiene is supplied to the second stage contacting zone only by the second stage feed. The second stage feed preferably is heated to a suitable temperature by heating means before the second stage contacting takes place. The heating means for the second stage feed may be, for example, one or more heat exchanger(s) or a heated inert filling. In order to maintain a temperature that is sufficient to enable the conversion of a mixture of ethanol and acetaldehyde to 1,3-butadiene, the temperature of the second stage feed preferably is higher than the temperature of the first stage effluent. Preferably, said temperature increase is provided between the first and the second stage contacting by suitable heating means. Said heating means may be, for example, one or more heat exchanger(s) or a heated inert filling (cf. below for more details).

Alternatively, the second stage contacting of the process for the production of 1,3-butadiene of the present invention takes place under isothermal conditions, i.e. the heat required for the conversion of the mixture of ethanol and acetaldehyde to 1,3-butadiene is supplied to the second stage contacting zone by heating means.

Further alternatively, the first and second stage contacting of the process for the production of 1,3-butadiene of the present invention take place under isothermal conditions, i.e. the heat required for the conversion of the mixture of ethanol and acetaldehyde to 1,3-butadiene is supplied to the first and second stage contacting zone by heating means.

Preferably, the temperature of the second stage feed at the inlet to the second stage contacting is in a range of from 200 to 400° C., preferably from 325 to 375° C.

According to a preferred embodiment, the first stage catalyst comprises, based on the total weight of the first stage catalyst, a total amount of element MA1 of from 0.02 to 14 wt. %, preferably from 0.02 to 12 wt. %, preferably from 0.04 to 6 wt. %, more preferably from 0.04 to 0.08 wt. %, calculated as elemental metal.

According to another preferred embodiment, the first stage catalyst comprises, based on the total weight of the first stage catalyst, a total amount of element MB1 of from 0.8 to 10 wt. %, preferably from 1.5 to 4 wt. %, more preferably from 1.5 to 3 wt. %, calculated as elemental metal.

According to another preferred embodiment, the second stage catalyst comprises, based on the total weight of the second stage catalyst, a total amount of element MB2 of from 0.8 to 10 wt. %, preferably from 1.5 to 4 wt. %, more preferably from 1.5 to 3 wt. %, calculated as elemental metal.

The process for the production of 1,3-butadiene according to the present invention is advantageous since the concept of coupling the one-step process (first stage contacting i)) with the second part of the two-step process (second stage contacting ii)) enables the production of 1,3-butadiene in high yields with a range of different catalysts. The first stage produces both acetaldehyde and 1,3-butadiene from the first stage feed comprising ethanol, thus the process may even be carried out with a first stage feed that is free of acetaldehyde. Moreover, the process is not restricted to the use of certain supports for the first and second stage catalysts, and may even be carried out with unsupported catalysts. The process of the invention is thus very versatile in terms of the first and second stage catalysts, i.e. the catalytically active species and supports that may be used.

The first and second stage catalysts need to be regenerated, and such regeneration may take place under adiabatic or isothermal conditions.

Further details regarding the second stage contacting of a second stage feed with a second stage catalyst under adiabatic conditions are set out in the application entitled “Adiabatically conducted process for the production of 1,3-butadiene from mixtures of ethanol and acetaldehyde” (PCT application number PCT/EP2022/058731, attorney reference SH 1655-02WO, filed on even date herewith), the disclosure of which application is incorporated herein in its entirety. Said application entitled “Adiabatically conducted process for the production of 1,3-butadiene from mixtures of ethanol and acetaldehyde” claims priority from European patent application EP21461531.2 filed on 1 Apr. 2021, which is also the filing date of EP 21461532.0 (from which the present application claims priority).

Further details regarding regeneration of the second stage catalyst under adiabatic conditions are set out in the application entitled “Adiabatically conducted process for the production of 1,3-butadiene from mixtures of ethanol and acetaldehyde with catalyst regeneration” (PCT application number PCT/EP2022/058716, attorney reference SH 1657-02WO, filed on even date herewith), the disclosure of which application is incorporated herein in its entirety. Said application entitled “Adiabatically conducted process for the production of 1,3-butadiene from mixtures of ethanol and acetaldehyde with catalyst regeneration” claims priority from European patent application EP 21461530.4 filed on 1 Apr. 2021, which is also the filing date of EP 21461532.0 (from which the present application claims priority).

In the process according to the invention, MA1 is preferably selected from the group consisting of zinc, copper, silver, chromium, magnesium, and nickel.

Alternatively, in the process according to the invention, MA1 is preferably selected from the group consisting of zinc, copper, silver, magnesium, cobalt and ruthenium.

More preferably, in the process according to the invention, MA1 is selected from the group consisting of zinc, copper and magnesium, more preferably from the group consisting of zinc and copper.

Most preferably, MA1 is zinc.

Most preferably, MA1 is copper.

Preferably, in the process according to the invention, the first stage catalyst comprises zinc.

MA1 catalyses the conversion of ethanol to acetaldehyde. Due to the presence of MA1 in the first stage catalyst, it is possible for the first stage feed to only comprise ethanol as 1,3-butadiene precursor and be free of acetaldehyde.

MA1 may be present in metallic form, as metal oxide and/or as metal sulfide. In the process according to the invention, MA1 is preferably present in an oxide form.

When MA1 is present in an oxide form, the catalyst advantageously does not have to be activated.

More preferably, the first stage catalyst comprises zinc oxide and/or copper oxide.

In the process according to the invention, MB1 is preferably selected from the group consisting of tantalum, zirconium, niobium, hafnium. More preferably, MB1 is tantalum.

Preferably, in the process according to the invention, the first stage catalyst comprises tantalum.

MB1 catalyses the conversion of mixtures of ethanol and acetaldehyde to 1,3-butadiene. Thus, due to the presence of MA1 and MB1, the first stage catalyst catalyses both the conversion of ethanol to acetaldehyde and the conversion of a mixture of ethanol and acetaldehyde to 1,3-butadiene.

In the process according to the invention, MB1 is preferably present in an oxide form. More preferably, the first stage catalyst comprises tantalum oxide.

It is particularly advantageous when the first stage catalyst comprises tantalum oxide since tantalum oxide shows the best catalytic results in the two-step process so far.

According to a preferred embodiment of the invention, the first stage catalyst comprises, based on the total weight of the first stage catalyst,
a) zinc oxide in an amount of from 0.05 to 18 wt. %, preferably from 0.05 to 5 wt. %, more preferably from 0.05 to 1 wt. %, more preferably from 0.05 to 0.5 wt. %, more preferably from 0.05 to 0.3 wt. %, more preferably from 0.05 to 0.1 wt. %, most preferably about 0.1 wt. %, calculated as ZnO, and/or
b) tantalum oxide in an amount of from 1 to 13 wt. %, preferably from 2 to 3 wt. %, more preferably about 2 wt. %, calculated as Ta₂O₅.

Most preferably, the first stage catalyst comprises, based on the total weight of the first stage catalyst,
a) zinc oxide in an amount of from 0.05 to 18 wt. %, preferably from 0.05 to 5 wt. %, more preferably from 0.05 to 1 wt. %, more preferably from 0.05 to 0.5 wt. %, more preferably from 0.05 to 0.3 wt. %, more preferably from 0.05 to 0.2 wt. %, most preferably about 0.1 wt. %, calculated as ZnO, and
b) tantalum oxide in an amount of from 1 to 13 wt. %, preferably from 2 to 3 wt. %, more preferably about 2 wt. %, calculated as Ta₂O₅.

According to a preferred embodiment of the invention, the first stage catalyst comprises, based on the total weight of the first stage catalyst,
a) copper oxide in an amount of from 0.05 to 30 wt. %, preferably from 0.05 to 15 wt. %, more preferably from 0.05 to 10 wt. %, most preferably from 0.05 to 5 wt. %, calculated as CuO, and/or
b) tantalum oxide in an amount of from 1 to 13 wt. %, preferably from 2 to 3 wt. %, more preferably about 2 wt. %, calculated as Ta₂O₅.

Most preferably, the first stage catalyst comprises, based on the total weight of the first stage catalyst,
a) copper oxide in an amount of from 0.05 to 30 wt. %, preferably from 0.05 to 15 wt. %, more preferably from 0.05 to 10 wt. %, most preferably from 0.05 to 5 wt calculated as CuO, and
b) tantalum oxide in an amount of from 1 to 13 wt. %, preferably from 2 to 3 wt. %, more preferably about 2 wt. %, calculated as Ta₂O₅.

Preferably, the first stage catalyst is a supported catalyst. More preferably, the support of the first stage catalyst is selected from the group consisting of ordered and non-ordered porous silica supports, aluminium oxide supports, aluminosilicate supports, clays, other porous oxide supports, and mixtures thereof.

Most preferably, the support of the first stage catalyst is a silica support, preferably an ordered or non-ordered porous silica support.

Supported catalysts are particularly advantageous, because they allow simple control of the concentration and dispersion of active sites, simple preparation of the catalyst by simple impregnation of any form and shape of the support, and easy access of the reacting molecules to all active sites of the catalyst.

Preferably, the support of the first stage catalyst has a specific surface area (SSA) in a range of from 130 to 550 m²/g, more preferably in a range of from 190 to 350 m²/g. Within the framework of the present text, the term “specific surface area” means the BET specific surface area (in m²/g) determined by the single-point BET method according to ISO 9277:2010, complemented by, if applicable, ISO 18757:2003.

Preferably, the support of the first stage catalyst has an average pore diameter in a range of from 30 to 300 Å (determined by the method of Barrett, Joyner and Halenda).

Preferably, the support of the first stage catalyst has a pore volume in a range of from 0.2 to 1.5 ml/g (determined by the method of Barrett, Joyner and Halenda).

More preferably, the support of the first stage catalyst is a silica support with a specific surface area in a range of from 130 to 550 m²/g, most preferably from 190 to 350 m²/g, and/or an average pore diameter in a range of from 30 to 300 Å, and/or a pore volume in a range of from 0.2 to 1.5 ml/g.

Most preferably, the support of the first stage catalyst is a silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

Most preferably, the support of the first stage catalyst is an ordered or non-ordered porous silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

In the process according to the invention, MB2 is preferably selected from the group consisting of tantalum, zirconium, niobium, hafnium, preferably MB2 is tantalum.

Preferably, the second stage catalyst comprises tantalum.

The second stage catalyst comprising MB2 catalyses the conversion of mixtures of ethanol and acetaldehyde to 1,3-butadiene. It is contacted with at least parts of the effluent of the first stage, whereby the second stage feed comprises ethanol and acetaldehyde. This process is particularly advantageous, because it increases the yield of 1,3-butadiene inter alia by conversion of acetaldehyde produced in the first stage and by conversion of ethanol that did not react in the first stage to 1,3-butadiene.

Preferably, MB2 is present in an oxide form. More preferably, the second stage catalyst comprises tantalum oxide.

According to a preferred embodiment, the second stage catalyst comprises tantalum oxide in an amount of from 1 to 13 wt. %, preferably from 1 to 11 wt. %, preferably from 2 to 3 wt. %, more preferably about 2 wt. %, calculated as Ta₂O₅, based on the total weight of the second stage catalyst.

Preferably, in the process according to the invention, MA1 is selected from the group consisting of zinc, copper, silver, magnesium, cobalt and ruthenium, MB1 is tantalum and MB2 is tantalum.

More preferably, in the process according to the invention, MA1 is selected from the group consisting of zinc and copper, MB1 is tantalum and MB2 is tantalum.

Most preferably, in the process according to the invention, the first stage catalyst comprises zinc and tantalum and the second stage catalyst comprises tantalum.

Most preferably, in the process according to the invention, the first stage catalyst comprises copper and tantalum and the second stage catalyst comprises tantalum.

Preferably, the second stage catalyst is a supported catalyst. More preferably, the support of the second stage catalyst is selected from the group consisting of ordered and non-ordered porous silica supports, aluminium oxide supports, aluminosilicate supports, clays, other porous oxide supports and mixtures thereof.

Most preferably, the support of the second stage catalyst is a silica support, preferably an ordered or non-ordered porous silica support.

Preferably, the support of the second stage catalyst has a specific surface area (SSA) in a range of from 130 to 550 m²/g, more preferably in a range of from 190 to 350 m²/g.

Preferably, the support of the second stage catalyst has an average pore diameter in a range of from 30 to 300 Å (determined by the method of Barrett, Joyner and Halenda).

Preferably, the support of the second stage catalyst has a pore volume in a range of from 0.2 to 1.5 ml/g (determined by the method of Barrett, Joyner and Halenda).

More preferably, the support of the second stage catalyst is a silica support with a specific surface area in a range of from 130 to 550 m²/g, most preferably from 190 to 350 m²/g, and/or an average pore diameter in a range of from 30 to 300 Å, and/or a pore volume in a range of from 0.2 to 1.5 ml/g.

Most preferably, the support of the second stage catalyst is a silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

Most preferably, the support of the second stage catalyst is an ordered or non-ordered porous silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

In the process according to the invention, preferably both the first and the second stage catalysts are supported catalysts.

More preferably, the support of both the first and the second stage catalyst is selected from the group consisting of ordered and non-ordered porous silica supports, aluminium oxide supports, aluminosilicate supports, clays, other porous oxide supports and mixtures thereof.

Most preferably, the support of both the first and the second stage catalyst is a silica support, preferably an ordered or non-ordered porous silica support.

Preferably, the support of both the first and the second stage catalyst has a specific surface area (SSA) in a range of from 130 to 550 m²/g, more preferably in a range of from 190 to 350 m²/g.

Preferably, the support of both the first and the second stage catalyst has an average pore diameter in a range of from 30 to 300 Å (determined by the method of Barrett, Joyner and Halenda).

Preferably, the support of both the first and the second stage catalyst has a pore volume in a range of from 0.2 to 1.5 ml/g (determined by the method of Barrett, Joyner and Halenda).

More preferably, the support of both the first and the second stage catalyst is a silica support with a specific surface area in a range of from 130 to 550 m²/g, most preferably from 190 to 350 m²/g, and/or an average pore diameter in a range of from 30 to 300 Å, and/or a pore volume in a range of from 0.2 to 1.5 ml/g.

Most preferably, the support of both the first and the second stage catalyst is a silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

Most preferably, the support of both the first and the second stage catalyst is an ordered or non-ordered porous silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

Preferably, the first stage catalyst comprises zinc and tantalum in a molar ratio of from 0.01 to 1.5, more preferably from 0.01 to 1, more preferably from 0.1 to 0.7, most preferably from 0.1 to 0.2.

The molar ratio of zinc and tantalum as defined is calculated based on elemental zinc and elemental tantalum (not to the respective oxides).

In the studies underlying the present invention, it was surprisingly found that even when zinc is present in a smaller molar amount than tantalum in the first stage catalyst, i.e. when the molar ratio of zinc to tantalum in the first stage catalyst is (significantly) smaller than 1, the process according to the invention still delivers a high yield of 1,3-butadiene.

In the process according to invention, the first stage feed preferably additionally comprises acetaldehyde.

Preferably, the acetaldehyde concentration is within a range of from 2 to 30 vol. %, more preferably from 5 to 20 vol. %, most preferably from 7 to 15 vol. %, each based on total volume of the first stage feed.

The addition of a small amount of acetaldehyde to the first stage feed is particularly advantageous, because it results in the production of 1,3-butadiene from the beginning of the first stage catalytic bed and therefore selectivity to 1,3-butadiene is increased.

According to a preferred embodiment, the acetaldehyde that is fed into the first stage is a recycled fraction of the first stage effluent, or the second stage effluent, or both the first and second stage effluent.

Preferably, the acetaldehyde concentration in the second stage feed is in a range of from 5 to 40 vol. %, more preferably from 10 to 30 vol. %, each based on total volume of the second stage feed.

In the process according to the invention, the first stage catalyst is preferably produced or producible according to a method comprising:
a) impregnating a first stage support (as defined herein) with a first stage catalyst precursor comprising an MA1 compound and an MB1 compound;
b) drying the impregnated first stage support; and
c) calcining the dried impregnated first stage support.

Preferably, the first stage catalyst is produced or producible according to a method comprising:
a) impregnating a first stage support comprising silica with a first stage catalyst precursor comprising an MA1 compound and an MB1 compound;
b) drying the impregnated first stage support; and
c) calcining the dried impregnated first stage support.

Preferably, the MA1 compound is selected from the group consisting of zinc compounds, copper compounds, silver compounds, gold compounds, chromium compounds, cerium compounds, magnesium compounds, platinum compounds, palladium compounds, cadmium compounds, iron compounds, manganese compounds, ruthenium compounds, cobalt compounds, and nickel compounds.

More preferably, the MA1 compound is selected from the group consisting of zinc compounds, copper compounds, chromium compounds, silver compounds, magnesium compounds, and nickel compounds.

More preferably, the MA1 compound is selected from the group consisting of zinc compounds, copper compounds, silver compounds, magnesium compounds, cobalt compounds, and ruthenium compounds.

More preferably, the MA1 compound is selected from the group consisting of zinc compounds, copper compounds, and magnesium compounds.

More preferably, the MA1 compound is selected from the group consisting of zinc compounds and copper compounds.

Most preferably, the MA1 compound is a zinc compound.

Most preferably, the MA1 compound is a copper compound.

Most preferably, the zinc compound is a zinc salt, preferably an organic or inorganic acid zinc salt.

According to a preferred embodiment, the zinc compound is selected from the group consisting of zinc acetate, zinc nitrate and zinc chloride.

Preferably, the MB1 compound is selected from the group consisting of tantalum compounds, zirconium compounds, niobium compounds, hafnium compounds, titanium compounds, and tin compounds.

More preferably, the MB1 compound is selected from the group consisting of tantalum compounds, zirconium compounds, niobium compounds, and hafnium compounds.

Most preferably, the MB1 compound is a tantalum compound.

Preferably, the first stage support to be impregnated in step a) is an ordered or non-ordered porous silica support.

More preferably, the first stage support to be impregnated in step a) is an ordered or non-ordered porous silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

Alternatively, in the process according to the invention, the first stage catalyst is preferably produced or producible according to a method comprising:
a) impregnating a first stage support (as defined herein) with a first stage catalyst precursor comprising an MA1 compound or an MB1 compound;
b) drying the impregnated first stage support;
c) calcining the dried impregnated first stage support;
d) impregnating the calcined dried impregnated first stage support with a first stage catalyst precursor comprising the other of an MA1 compound and an MB1 compound;
e) drying the impregnated calcined dried impregnated first stage support; and
f) calcining the dried impregnated calcined dried impregnated first stage support.

Preferably, the first stage catalyst is produced or producible according to a method comprising:
a) impregnating a first stage support comprising silica with a first stage catalyst precursor comprising an MA1 compound or an MB1 compound;
b) drying the impregnated first stage support;
c) calcining the dried impregnated first stage support;
d) impregnating the calcined dried impregnated first stage support with a first stage catalyst precursor comprising the other of an MA1 compound and an MB1 compound;
e) drying the impregnated calcined dried impregnated first stage support; and
f) calcining the dried impregnated calcined dried impregnated first stage support.

Preferably, the MA1 compound is selected from the group consisting of zinc compounds, copper compounds, silver compounds, gold compounds, chromium compounds, cerium compounds, magnesium compounds, platinum compounds, palladium compounds, cadmium compounds, iron compounds, manganese compounds, ruthenium compounds, cobalt compounds, and nickel compounds.

More preferably, the MA1 compound is selected from the group consisting of zinc compounds, copper compounds, chromium compounds, silver compounds, magnesium compounds, and nickel compounds.

More preferably, the MA1 compound is selected from the group consisting of zinc compounds, copper compounds, silver compounds, magnesium compounds, cobalt compounds, and ruthenium compounds.

More preferably, the MA1 compound is selected from the group consisting of zinc compounds, copper compounds, and magnesium compounds.

More preferably, the MA1 compound is selected from the group consisting of zinc compounds and copper compounds.

Most preferably, the MA1 compound is a zinc compound.

Most preferably, the MA1 compound is a copper compound.

Preferably, the MB1 compound is selected from the group consisting of tantalum compounds, zirconium compounds, niobium compounds, hafnium compounds, titanium compounds, and tin compounds.

More preferably, the MB1 compound is selected from the group consisting of tantalum compounds, zirconium compounds, niobium compounds, and hafnium compounds.

Most preferably, the MB1 compound is a tantalum compound.

Preferably, the first stage support to be impregnated in step a) is an ordered or non-ordered porous silica support.

More preferably, the first stage support to be impregnated in step a) is an ordered or non-ordered porous silica support with a specific surface area in a range of from 130 to 550 m²/g, most preferably from 190 to 350 m²/g, and an average pore diameter in a range of from 30 to 300 Å, and a pore volume in a range of from 0.2 to 1.5 ml/g.

The described production methods for the first stage catalyst are particularly advantageous since they enable a straightforward production of a versatile range of different first stage catalysts.

Another preferred embodiment of the present invention relates to a process for the production of 1,3-butadiene, the process comprising the following stages
i) a first stage contacting of a first stage feed comprising ethanol with a first stage catalyst,
   wherein the first stage catalyst comprises element MA1 and element MB1,
      MA1 is selected from the group consisting of zinc, copper, silver, gold, chromium, cerium, magnesium, platinum, palladium, cadmium, iron, manganese, ruthenium, cobalt, and nickel, and
      MB1 is selected from the group consisting of tantalum, zirconium, niobium, hafnium, titanium, and tin,
   to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises element MB2, and
      MB2 is selected from the group consisting of tantalum, zirconium, niobium, hafnium, titanium, and tin,
   to produce a second stage effluent comprising 1,3-butadiene,
wherein the second stage catalyst does not comprise any element MA1 selected from the group consisting of zinc, copper, silver, gold, chromium, cerium, magnesium, platinum, palladium, cadmium, iron, manganese, ruthenium, cobalt, and nickel.

Another preferred embodiment of the present invention relates to a process for the production of 1,3-butadiene, the process comprising the following stages
i) a first stage contacting of a first stage feed comprising ethanol with a first stage catalyst,
   wherein the first stage catalyst comprises element MA1 and element MB1,
      MA1 is selected from the group consisting of zinc, copper, silver, magnesium, ruthenium, and cobalt, and
      MB1 is tantalum,
   to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises element MB2, and MB2 is tantalum, to produce a second stage effluent comprising 1,3-butadiene,
wherein the second stage catalyst does not comprise any element MA1 selected from the group consisting of zinc, copper, silver, magnesium, ruthenium, and cobalt.

Another preferred embodiment of the present invention relates to a process for the production of 1,3-butadiene, the process comprising the following stages
i) a first stage contacting of a first stage feed comprising ethanol with a first stage catalyst,
   wherein the first stage catalyst comprises element MA1 and element MB1,
      MA1 is selected from the group consisting of zinc and copper, and MB1 is tantalum,
   to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises element MB2, and MB2 is tantalum, to produce a second stage effluent comprising 1,3-butadiene,
wherein the second stage catalyst does not comprise any element MA1 selected from the group consisting of zinc and copper.

Another preferred embodiment of the present invention relates to a process for the production of 1,3-butadiene, the process comprising the following stages
i) a first stage contacting of a first stage feed comprising ethanol with a first stage catalyst,
   wherein the first stage catalyst comprises element MA1 and element MB1, wherein MA1 is zinc and MB1 is tantalum,
   to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises element MB2, wherein MB2 is tantalum,
   to produce a second stage effluent comprising 1,3-butadiene,
wherein the second stage catalyst does not comprise any zinc.

Another preferred embodiment of the present invention relates to a process for the production of 1,3-butadiene, the process comprising the following stages
i) a first stage contacting of a first stage feed comprising ethanol with a first stage catalyst,
   wherein the first stage catalyst comprises element MA1 and element MB1, wherein MA1 is copper and MB1 is tantalum,
   to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises element MB2, wherein MB2 is tantalum, to produce a second stage effluent comprising 1,3-butadiene,
wherein the second stage catalyst does not comprise any copper.

Another preferred specific embodiment of the present invention relates to a process for the production of 1,3-butadiene, the process comprising the following stages
i) a first stage contacting of a first stage feed comprising ethanol with a first stage catalyst,
   wherein the first stage catalyst comprises zinc oxide and tantalum oxide, to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises tantalum oxide,
   to produce a second stage effluent comprising 1,3-butadiene,
wherein the second stage catalyst does not comprise any zinc oxide.

Another preferred specific embodiment of the present invention relates to a process for the production of 1,3-butadiene, the process comprising the following stages
i) a first stage contacting of a first stage feed comprising ethanol with a first stage catalyst,
   wherein the first stage catalyst comprises copper oxide and tantalum oxide, to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde,
   wherein the second stage catalyst comprises tantalum oxide,
   to produce a second stage effluent comprising 1,3-butadiene,
wherein the second stage catalyst does not comprise any copper oxide.

Another preferred embodiment of the present invention relates to a process for the production of 1,3-butadiene, the process comprising the following stages
i) a first stage contacting of a first stage feed comprising ethanol with a first stage catalyst, wherein the first stage catalyst comprises element MA1 and element MB1, wherein MA1 is zinc and MB1 is tantalum, to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde, wherein the second stage catalyst comprises element MB2, wherein MB2 is tantalum, to produce a second stage effluent comprising 1,3-butadiene,
wherein the first stage catalyst does not comprise any of magnesium, calcium, barium, cerium and tin,
and preferably wherein the second stage catalyst does not comprise any zinc.

Another preferred embodiment of the present invention relates to a process for the production of 1,3-butadiene, the process comprising the following stages
i) a first stage contacting of a first stage feed comprising ethanol with a first stage catalyst, wherein the first stage catalyst comprises element MA1 and element MB1, wherein MA1 is copper and MB1 is tantalum, to produce a first stage effluent comprising acetaldehyde and 1,3-butadiene,
ii) a second stage contacting of a second stage feed with a second stage catalyst, the second stage feed comprising at least part of the first stage effluent, and the second stage feed comprising ethanol and acetaldehyde, wherein the second stage catalyst comprises element MB2, wherein MB2 is tantalum, to produce a second stage effluent comprising 1,3-butadiene,
wherein the first stage catalyst does not comprise any of magnesium, calcium, barium, cerium and tin,
and preferably wherein the second stage catalyst does not comprise any copper.

### 2) Catalyst System

According to another aspect, the invention relates to a catalyst system for use in the production of 1,3-butadiene from ethanol comprising
i) a first stage catalyst comprising element MA1 and element MB1, wherein
   MA1 is selected from the group consisting of zinc, copper, silver, gold, chromium, cerium, magnesium, platinum, palladium, cadmium, iron, manganese, ruthenium, cobalt, and nickel, and
   MB1 is selected from the group consisting of tantalum, zirconium, niobium, hafnium, titanium, and tin, and
ii) a second stage catalyst comprising element MB2, wherein
   MB2 is selected from the group consisting of tantalum, zirconium, niobium, hafnium, titanium, and tin.

According to one embodiment of the catalyst system according to the invention, element MB1 of the first stage catalyst is the same as element MB2 of the second stage catalyst.

According to another embodiment of the catalyst system according to the invention, element MB1 of the first stage catalyst is different from element MB2 of the second stage catalyst.

According to a preferred embodiment, the first stage catalyst of the catalyst system according to the invention comprises, based on the total weight of the first stage catalyst, a total amount of element MA1 of from 0.02 to 14 wt. %, preferably from 0.04 to 6 wt. %, more preferably from 0.04 to 0.08 wt. %, calculated as elemental metal.

According to another preferred embodiment, the first stage catalyst of the catalyst system according to the invention comprises, based on the total weight of the first stage catalyst, a total amount of element MB1 of from 0.8 to 10 wt. %, preferably from 1.5 to 4 wt. %, more preferably from 1.5 to 3 wt. %, calculated as elemental metal.

According to another preferred embodiment, the second stage catalyst of the catalyst system according to the invention comprises, based on the total weight of the second stage catalyst, a total amount of element MB2 of from 0.8 to 10 wt. %, preferably from 1.5 to 4 wt. %, more preferably from 1.5 to 3 wt. %, calculated as elemental metal.

In one embodiment of the catalyst system according to the invention, the first and the second stage catalyst are in contact with one another in a single packing (i.e. inside the same reactor).

In another embodiment, the first and the second stage catalysts are separated by an inert filling in a single packing (i.e. inside the same reactor).

Preferably, the inert filling is selected from the group consisting of silicon carbide, inert ceramic beds, ceramic beads, extrudates, rings with diameter of 2-7 mm, stainless steel mesh, foams, and mixtures thereof.

In another embodiment, the first and the second stage catalysts are located in separate reactors that are connected with one another in series.

Preferably, in the catalyst system according to the invention, MA1 is selected from the group consisting of zinc, copper, silver, chromium, magnesium, and nickel.

Preferably, in the catalyst system according to the invention, MA1 is selected from the group consisting of zinc, copper, silver, magnesium, cobalt and ruthenium.

More preferably, MA1 is selected from the group consisting of zinc, copper and magnesium.

More preferably, MA1 is selected from the group consisting of zinc and copper.

Most preferably, MA1 is zinc.

Most preferably, MA1 is copper.

Preferably, the first stage catalyst comprises zinc.

Preferably, the first stage catalyst comprises copper.

According to one embodiment of the catalyst system according to the invention, MA1 is present in an oxide form.

Most preferably, the first stage catalyst comprises zinc oxide.

Most preferably, the first stage catalyst comprises copper oxide.

Preferably, in the catalyst system according to the invention, MB1 is selected from the group consisting of tantalum, zirconium, niobium, hafnium. Most preferably, MB1 is tantalum.

Preferably, the first stage catalyst comprises tantalum.

According to one embodiment of the catalyst system according to the invention, MB1 is present in an oxide form. Most preferably, the first stage catalyst comprises tantalum oxide.

Preferably, in the catalyst system according to the invention, the first stage catalyst comprises zinc and tantalum.

Preferably, in the catalyst system according to the invention, the first stage catalyst comprises copper and tantalum.

More preferably, in the catalyst system according to the invention, the first stage catalyst comprises zinc oxide and tantalum oxide.

More preferably, in the catalyst system according to the invention, the first stage catalyst comprises copper oxide and tantalum oxide.

Preferably, in the catalyst system according to the invention, MA1 is selected from the group consisting of zinc, copper, silver, magnesium, cobalt and ruthenium, MB1 is tantalum and MB2 is tantalum. More preferably, in the catalyst system according to the invention, MA1 is selected from the group consisting of zinc and copper, MB1 is tantalum and MB2 is tantalum.

Preferably, in the catalyst system according to the invention, the first stage catalyst comprises zinc and tantalum, and the second stage catalyst comprises tantalum.

Preferably, in the catalyst system according to the invention, the first stage catalyst comprises copper and tantalum, and the second stage catalyst comprises tantalum.

Preferably, in the catalyst system according to the invention, the first stage catalyst comprises zinc oxide and tantalum oxide, and the second stage catalyst comprises tantalum oxide.

Preferably, in the catalyst system according to the invention, the first stage catalyst comprises copper oxide and tantalum oxide, and the second stage catalyst comprises tantalum oxide.

According to a preferred embodiment of the catalyst system according to the invention, the first stage catalyst comprises
zinc oxide, preferably in an amount of from 0.05 to 18 wt. %, more preferably from 0.05 to 5 wt. %, more preferably from 0.05 to 1 wt. %, more preferably from 0.05 to 0.5 wt. %, more preferably from 0.05 to 0.3 wt. %, more preferably from 0.05 to 0.2 wt. %, most preferably about 0.1 wt. %, calculated as ZnO, and/or
tantalum oxide, preferably in an amount of from 1 to 13 wt. %, more preferably from 2 to 3 wt. %, most preferably about 2 wt. %, calculated as Ta₂O₅, each based on the total weight of the first stage catalyst.

According to another preferred embodiment of the catalyst system according to the invention, the first stage catalyst comprises
zinc oxide in an amount of from 0.05 to 18 wt. %, more preferably from 0.05 to 5 wt. %, more preferably from 0.05 to 1 wt. %, more preferably from 0.05 to 0.5 wt. %, more preferably from 0.05 to 0.3 wt. %, more preferably from 0.05 to 0.2 wt. %, most preferably about 0.1 wt. %, calculated as ZnO, and
tantalum oxide in an amount of from 1 to 13 wt. %, more preferably from 2 to 3 wt. %, most preferably about 2 wt. %, calculated as Ta₂O₅, each based on the total weight of the first stage catalyst.

According to a preferred embodiment of the catalyst system according to the invention, the first stage catalyst comprises
copper oxide, preferably in an amount of from 0.05 to 30 wt. %, more preferably from 0.05 to 15 wt. %, most preferably from 0.05 to 10 wt calculated as CuO, and/or
tantalum oxide, preferably in an amount of from 1 to 13 wt. %, more preferably from 2 to 3 wt. %, most preferably about 2 wt. %, calculated as Ta₂O₅, each based on the total weight of the first stage catalyst.

According to another preferred embodiment of the catalyst system according to the invention, the first stage catalyst comprises
copper oxide in an amount of from 0.05 to 30 wt. %, more preferably from 0.05 to 15 wt. %, most preferably from 0.05 to 10 wt, calculated as CuO, and
tantalum oxide in an amount of from 1 to 13 wt. %, more preferably from 2 to 3 wt. %, most preferably about 2 wt. %, calculated as Ta₂O₅, each based on the total weight of the first stage catalyst.

According to another preferred embodiment of the catalyst system according to the invention, the first stage catalyst is a supported catalyst.

According to yet another preferred embodiment of the catalyst system according to the invention, the second stage catalyst is a supported catalyst.

Preferably, both the first and the second stage catalyst are supported catalysts.

Preferably, the support of the first stage catalyst, of the second stage catalyst, or of both the first and second stage catalyst is selected from the group consisting of ordered and non-ordered porous silica supports, aluminium oxide supports, aluminosilicate supports, clays, other porous oxide supports and mixtures thereof.

Most preferably, the support of the first stage catalyst is an ordered or non-ordered porous silica support.

Most preferably, the support of the second stage catalyst is an ordered or non-ordered porous silica support.

Most preferably, the support of both the first and second stage catalyst is an ordered or non-ordered porous silica support.

Another embodiment of the present invention relates to a catalyst system for use in the production of 1,3-butadiene from ethanol, wherein the second stage catalyst does not comprise any zinc, preferably does not comprise any element MA1, MA1 being selected from the group consisting of zinc, copper, silver, gold, chromium, cerium, magnesium, platinum, palladium, cadmium, iron, manganese, ruthenium, cobalt, and nickel.

Another embodiment of the present invention relates to a catalyst system for use in the production of 1,3-butadiene from ethanol, wherein the second stage catalyst does not comprise any zinc, preferably does not comprise any element MA1, MA1 being selected from the group consisting of zinc, copper, silver, magnesium, ruthenium, and cobalt.

Another embodiment of the present invention relates to a catalyst system for use in the production of 1,3-butadiene from ethanol, wherein the second stage catalyst does not comprise any copper, preferably does not comprise any element MA1, MA1 being selected from the group consisting of zinc, copper, silver, gold, chromium, cerium, magnesium, platinum, palladium, cadmium, iron, manganese, ruthenium, cobalt, and nickel.

Another embodiment of the present invention relates to a catalyst system for use in the production of 1,3-butadiene from ethanol, wherein the second stage catalyst does not comprise any copper, preferably does not comprise any element MA1, MA1 being selected from the group consisting of zinc, copper, silver, magnesium, ruthenium, and cobalt.

Another specific embodiment of the invention present relates to a catalyst system for use in the production of 1,3-butadiene from ethanol comprising
i) a first stage catalyst comprising zinc oxide and tantalum oxide, and
ii) a second stage catalyst comprising tantalum oxide,
wherein the second stage catalyst does not comprise any zinc oxide.

Another specific embodiment of the invention present relates to a catalyst system for use in the production of 1,3-butadiene from ethanol comprising
i) a first stage catalyst comprising copper oxide and tantalum oxide, and
ii) a second stage catalyst comprising tantalum oxide,
wherein the second stage catalyst does not comprise any copper oxide.

Another embodiment of the present invention relates to a catalyst system for use in the production of 1,3-butadiene from ethanol comprising
i) a first stage catalyst comprising element MA1 and element MB1, wherein MA1 is zinc and MB1 is tantalum, and
ii) a second stage catalyst comprising element MB2, wherein MB2 is tantalum,
wherein the first stage catalyst does not comprise any of magnesium, calcium, barium, cerium and tin.

Another embodiment of the present invention relates to a catalyst system for use in the production of 1,3-butadiene from ethanol comprising
i) a first stage catalyst comprising element MA1 and element MB1, wherein MA1 is copper and MB1 is tantalum, and
ii) a second stage catalyst comprising element MB2, wherein MB2 is tantalum,
wherein the first stage catalyst does not comprise any of magnesium, calcium, barium, cerium and tin.

Another embodiment of the present invention relates to a catalyst system for use in the production of 1,3-butadiene from ethanol comprising
i) a first stage catalyst comprising element MA1 and element MB1, wherein MA1 is zinc and MB1 is tantalum, and
ii) a second stage catalyst comprising element MB2, wherein MB2 is tantalum,
wherein the first stage catalyst does not comprise any of magnesium, calcium, barium, cerium and tin, and wherein the second stage catalyst does not comprise any zinc.

Another embodiment of the present invention relates to a catalyst system for use in the production of 1,3-butadiene from ethanol comprising
i) a first stage catalyst comprising element MA1 and element MB1, wherein MA1 is copper and MB1 is tantalum, and
ii) a second stage catalyst comprising element MB2, wherein MB2 is tantalum,
wherein the first stage catalyst does not comprise any of magnesium, calcium, barium, cerium and tin, and wherein the second stage catalyst does not comprise any copper.

### 3) Use of the Catalyst System

In another aspect, the invention relates to the use of a catalyst system as defined herein for the production of 1,3-butadiene from a feed comprising ethanol, preferably to decrease the required amount of acetaldehyde in the first stage feed or to dispense altogether with acetaldehyde in the first stage feed.

As described above, the first stage catalyst of the catalyst system according to the invention produces both acetaldehyde and 1,3-butadiene from the first stage feed comprising ethanol. At least parts of the effluent of the first stage are then contacted with the second stage catalyst, which increases the yield of 1,3-butadiene inter alia by conversion of acetaldehyde produced in the first stage and by conversion of ethanol that did not react in the first stage to 1,3-butadiene.

Thus, if desired, the process for the production of 1,3-butadiene according to the invention may be carried out with a first stage feed that is free of acetaldehyde or only contains a small amount of acetaldehyde.

### 4) Plant

In another aspect, the present invention relates to a plant comprising the catalyst system as defined herein.

Preferably, the plant according to the invention contains the catalyst system according to the invention in one reactor.

As described above, the first and the second stage catalyst of the catalyst system may be separated by an inert filling (as described above) in a single packing, i.e. inside the same reactor.

Preferably, said reactor is a continuous flow fixed bed reactor.

According to a preferred embodiment, at least part of said inert filling is heated by heating means. Said embodiment is particularly advantageous, because it allows an increase of the temperature of the second stage feed compared to the temperature of the first stage effluent, if desired.

Alternatively, in the plant according to the invention, the first stage catalyst and the second stage catalyst of the catalyst system according to the invention are contained in separate reactors that are connected in series.

Preferably, said reactors are continuous flow fixed bed reactors.

More preferably, heating means are contained in the connection between the first reactor containing the first stage catalyst and the second reactor containing the second stage catalyst. The heating means may be, for example, one or more heat exchanger(s). This allows an increase of the temperature of the second stage feed compared to the temperature of the first stage effluent, if desired.

Preferred embodiments of the process for the production of 1,3-butadiene according to the first aspect of the invention correspond to or can be derived from preferred embodiments of the catalyst system according to the second aspect of the invention or vice versa. Moreover, preferred embodiments of the process according to the invention correspond to or can be derived from preferred embodiments of the use of the catalyst system according to the invention or the plant according to the invention which are explained above or vice versa.

The following examples show the advantages of the present invention. Unless noted otherwise, all percentages are given by weight.

### EXAMPLES

### Example 1

A first stage catalyst comprising 3 wt. % Ta₂O₅ and 0.5 wt. % ZnO, based on the total weight of the first stage catalyst, on a SiO₂ support (3 wt. % Ta₂O₅-0.5 wt. % ZnO/SiO₂) was prepared as follows (one-step synthesis of catalyst):

45 g of support (SiO₂, CARiACT Q10) are impregnated with 50 mL of a methanolic solution containing 2.27 g of tantalum pentachloride and 0.85 g of zinc nitrate hexahydrate. The impregnated silica is dried at 140° C. for 6 hours, and subsequently calcined at 500° C. for 5 hours.

2 g of the obtained first stage catalyst are placed in a continuous flow stainless steel reactor. The reactor is heated to achieve 350° C. in the first and second stage contacting zones with 20 ml/min of nitrogen flow. The reaction is carried out using aqueous 96% ethanol as a first stage feed with a weight hourly space velocity (WHSV) of 1 h⁻¹. The results are calculated as follows:

$$Conversion = \frac{mass of the converted reactant}{mass of the feed} \cdot 100 \%$$

$$Selectivity = \frac{C mols in butadiene}{C mols in all products} \cdot 100 \%$$

$$Yield = \frac{Conversion \cdot Selectivity}{100 \%}$$

### Example 2

The reaction is carried out as in Example 1 except that the first stage feed further contains 20 vol. % of acetaldehyde.

### Example 3

The reaction is carried out as in Example 1 except that the first stage catalyst 3 wt. % Ta₂O₅-0.5 wt. % ZnO/SiO₂ is prepared as follows (two-step synthesis of catalyst):

45 g of support (SiO₂, CARiACT Q10) are impregnated with 50 mL of a methanolic solution containing 2.27 g of tantalum pentachloride. Impregnated silica is dried at 140° C. for 6 hours, and subsequently calcined at 500° C. for 5 hours. Then it is impregnated with 50 mL of a methanolic solution containing 0.85 g of zinc nitrate hexahydrate, dried at 140° C. for 6 hours, and calcined at 500° C. for 5 hours.

### Example 4

The reaction is carried out as in Example 2 except that the catalyst is prepared in two steps as described in Example 3.

### Example 5

The reaction is carried out as in Example 1 except that the first stage feed further contains 10 vol. % of acetaldehyde.

### Example 6

The reaction is carried out as in Example 1 except that the first stage feed further contains 15 vol. % of acetaldehyde.

### Example 7

The reaction is carried out as in Example 3 except that the catalyst contains 2 wt. % of tantalum oxide, calculated as Ta₂O₅, based on the total weight of the first stage catalyst.

### Example 8

The reaction is carried out as in Example 7 except that the first stage feed further contains 5 vol. % of acetaldehyde.

### Example 9

The reaction is carried out as in Example 7 except that the first stage feed further contains 10 vol. % of acetaldehyde.

### Example 10

The reaction is carried out as in example 1 except that the first stage catalyst contains 0.25 wt. % of zinc oxide, calculated as ZnO, and 2 wt. % of tantalum oxide, calculated a Ta₂O₅, based on the total weight of the first stage catalyst. The WHSV is 0.7 h⁻¹.

### Example 11

The reaction is carried out as in example 10 except that the WHSV is 1 h⁻¹.

### Example 12

The reaction is carried out as in Example 10 except that the reaction temperature is 375° C.

### Example 13

The reaction is carried out as in Example 11 except that the reaction temperature is 375° C.

### Example 14 (According to the Invention)

The reaction is carried out as in Example 1 except the reactor is loaded with a first stage catalyst and a second stage catalyst:

The first stage catalyst is 1.4 g of ZnOₓ—TaOₓ/SiO₂. The concentration of ZnOₓ, calculated as ZnO, in the first stage catalyst is 0.1 wt. % based on the total weight of the first stage catalyst. The concentration of TaOₓ, calculated as Ta₂O₅, in the first stage catalyst is 2 wt. % based on the total weight of the first stage catalyst.

The second stage catalyst is 0.4 g of TaOₓ/SiO₂. The concentration of TaOₓ, calculated as Ta₂O₅, in the second stage catalyst is 2 wt. % based on the total weight of the second stage catalyst.

Both the first and the second stage catalyst are prepared by the one-step synthesis of catalyst as described above.

The obtained first and second stage catalyst are packed in contact with one another in a single packing (i.e. inside the same reactor) without any inert filling between the two stages.

The entire first stage effluent is fed into the second stage. The WHSV is 0.54 h⁻¹.

### Example 15

The reaction is carried out as in Example 1 except that reactor is loaded with tantalum oxide supported on silica (a second stage catalyst). The concentration of TaOₓ, calculated as Ta₂O₅, in said second stage catalyst is 3 wt. % based on the total weight of the second stage catalyst. The reaction temperature is 350° C. and the WHSV is 1 h⁻¹.

### Example 16

The reaction is carried out as in Example 1 except that the first stage catalyst contains 5 wt. % of zinc oxide, calculated as ZnO, and 2 wt. % of tantalum oxide, calculated a Ta₂O₅, based on the total weight of the first stage catalyst.

### Example 17

The reaction is carried out as in Example 16 except that the first stage feed further contains 10 vol. % of acetaldehyde.

The results of the 1,3-butadiene production processes for Examples 1 to 17 are summarized in Table 1 below.

TABLE 1

| | | | | Synthesis | | Reaction conditions | | | | 1,3-BD | |
|---|---|---|---|---|---|---|---|---|---|---|---|
| | | Loading [wt. %]¹ | | of | | T | WHSV | [AcH] | C | S | Y |
| Ex. | Catalyst | ZnO | Ta₂O₅ | catalyst | | [° C.] | [h⁻¹] | [vol. %] | [%] | [%] | [%] |
| 1 | ZnOₓ—TaOₓ/SiO₂ | 0.5 | 3 | 1 | step | 350 | 1 | 0 | 59 | 57 | 34 |
| 2 | ZnOₓ—TaOₓ/SiO₂ | 0.5 | 3 | 1 | step | 350 | 1 | 20 | 54 | 78 | 42 |
| 3 | ZnOₓ—TaOₓ/SiO₂ | 0.5 | 3 | 2 | steps | 350 | 1 | 0 | 60 | 55 | 33 |
| 4 | ZnOₓ—TaOₓ/SiO₂ | 0.5 | 3 | 2 | steps | 350 | 1 | 20 | 55 | 78 | 43 |
| 5 | ZnOₓ—TaOₓ/SiO₂ | 0.5 | 3 | 1 | step | 350 | 1 | 10 | 56 | 77 | 43 |
| 6 | ZnOₓ—TaOₓ/SiO₂ | 0.5 | 3 | 1 | step | 350 | 1 | 15 | 54 | 76 | 41 |
| 7 | ZnOₓ—TaOₓ/SiO₂ | 0.5 | 2 | 2 | steps | 350 | 1 | 0 | 60 | 61 | 37 |
| 8 | ZnOₓ—TaOₓ/SiO₂ | 0.5 | 2 | 2 | steps | 350 | 1 | 5 | 51 | 76 | 39 |
| 9 | ZnOₓ—TaOₓ/SiO₂ | 0.5 | 2 | 2 | steps | 350 | 1 | 10 | 52 | 77 | 40 |
| 10 | ZnOₓ—TaOₓ/SiO₂ | 0.25 | 2 | 1 | step | 350 | 0.7 | 0 | 69 | 56 | 39 |
| 11 | ZnOₓ—TaOₓ/SiO₂ | 0.25 | 2 | 1 | step | 350 | 1 | 0 | 53 | 49 | 26 |
| 12 | ZnOₓ—TaOₓ/SiO₂ | 0.25 | 2 | 1 | step | 375 | 0.7 | 0 | 89 | 59 | 53 |
| 13 | ZnOₓ—TaOₓ/SiO₂ | 0.25 | 2 | 1 | step | 375 | 1 | 0 | 80 | 54 | 43 |
| 14 | ZnOₓ—TaOₓ/SiO₂ | * | ** | 1 | step | 350 | 0.54 | 0 | 82 | 69 | 57 |
| | (first stage) | | | | | | | | | | |
| | TaOₓ/SiO₂ (second stage) | | | | | | | | | | |
| 15 | TaOₓ/SiO₂ | — | 3 | 1 | step | 350 | 1 | 0 | 0 | 0 | 0 |
| 16 | ZnOₓ—TaOₓ/SiO₂ | 5 | 2 | 2 | step | 350 | 1 | 0 | 77 | 50 | 39 |
| 17 | ZnOₓ—TaOₓ/SiO₂ | 5 | 2 | 2 | step | 350 | 1 | 10 | 63 | 69 | 44 |

T—temperature;
[AcH]—concentration of acetaldehyde in the (first stage) feed;
C—conversion of ethanol (or ethanol and acetaldehyde mixture);
S—selectivity;
Y—yield;
1,3-BD—1,3-butadiene;
* concentration of ZnOₓ, calculated as ZnO, in the first stage catalyst is 0.1 wt. % based on the total weight of the first stage catalyst;
** concentration of TaOₓ, calculated as Ta₂O₅, both in the first and the second stage catalyst is 2 wt. % based on the total weight of the first and second stage catalyst, respectively
¹The numbers are wt. % of metal oxide as indicated based on the total weight of the catalyst (for examples 1-13 and 15-17)

Examples 1 to 13 (with a first stage catalyst only), Example 15 (with a second stage catalyst only) and Examples 16 and 17 (with a first stage catalyst only) are comparative.

Example 15 shows that a TaOₓ/SiO₂ catalyst is inactive in the production of 1,3-butadiene without acetaldehyde in the feed. Looking at Comparative Examples 1 and 15, it is apparent that doping the silica-supported tantalum catalyst with zinc (cf. Example 1) allows the production of 1,3-butadiene from first stage feeds not comprising any acetaldehyde, i.e. doping with zinc allows the transition from a two-step to a one-step process. Further, addition of acetaldehyde to the feed (cf. Examples 2, 4 to 6, 8, and 9) gives, in comparison to Example 1 without co-fed acetaldehyde, a moderate decrease of conversion, but a marked increase in selectivity to 1,3-butadiene, and therefore leads to an increase in 1,3-butadiene yield. To increase the selectivity to 1,3-butadiene, lower amounts of acetaldehyde in comparison with undoped tantalum catalysts are required.

Looking at Examples 1 and 3 (and Examples 2 and 4), first stage catalyst synthesis in two steps does not affect conversion, selectivity or yield, when using tantalum in an amount of 3 wt. % (calculated as Ta₂O₅), as compared to one-step catalyst synthesis. Hence, the first stage catalyst doped with zinc works both in case zinc is loaded onto the support together with tantalum and also if they are loaded separately.

Looking at Examples 1, 3 and 7, lower tantalum loading of the catalyst does increase selectivity and therefore yield, when using tantalum in an amount of 2 wt. % (calculated as Ta₂O₅) as in Example 7, as compared to one-step catalyst synthesis (cf. Example 1), or two-step catalyst synthesis (cf. Example 3), each using tantalum in an amount of 3 wt. %.

Looking at Examples 7 to 9, each using tantalum in an amount of 2 wt. %, adding acetaldehyde in an amount of 5 vol. % to the first stage ethanol feed somewhat decreases conversion, but in view of increased selectivity, the yield of 1,3-butadiene is increased. Further increasing the amount of acetaldehyde in the first stage feed to 10 vol. %, as in Example 9, does not affect conversion, selectivity to, and yield of 1,3-butadiene compared to an addition of 5 vol. % of acetaldehyde (cf. Example 8). Hence, the addition of small amounts of acetaldehyde to the first stage feed improves selectivity to 1,3-butadiene. It is suspected that during a reaction without acetaldehyde, the upper part of the first stage catalyst mainly converts ethanol to acetaldehyde. When a high enough concentration of acetaldehyde is reached, the remaining catalytic bed of the first stage catalyst converts the mixture of ethanol and acetaldehyde to 1,3-butadiene. The addition of a small amount of acetaldehyde to the first stage feed results in the production of 1,3-butadiene from the beginning of the first stage contacting and, therefore, selectivity to 1,3-butadiene is increased.

Example 10 with lower zinc loading and at a lower WHSV compared to Example 1 shows better conversion and therefore yield of 1,3-butadiene, whereas a higher WHSV at low zinc loading (cf. Example 11) decreases both conversion and selectivity to (and therefore yield of) 1,3-butadiene. Therefore, a lower WHSV in the case of zinc doped first stage catalyst is preferred.

Example 12 with low zinc loading and at a low WHSV shows that a reaction temperature increase to 375° C. results in higher selectivity to 1,3-butadiene and much higher conversion, as compared to Example 10 at 350° C., so the yield of 1,3-butadiene is markedly increased. Higher temperatures favour the conversion of ethanol to both acetaldehyde and 1,3-butadiene. It is suspected that at higher temperatures a shorter part of the catalytic bed of the first stage catalyst is necessary to produce acetaldehyde and therefore a longer part of the catalytic bed can produce 1,3-butadiene. Similar to the results at lower temperature (cf. Examples 10 and 11 at 350° C.), a higher reaction temperature of 375° C., WHSV increase at low zinc loading (as in Example 13) decreases both conversion and selectivity to (and therefore yield of) 1,3-butadiene (as compared to Example 12).

Looking at Examples 7 and 16, when increasing the amount of zinc in the catalyst using the same amount of tantalum (2 wt. %) a major increase of conversion and a major decrease of selectivity to 1,3-butadiene are observed. Comparing Examples 16 and 17, each using tantalum in an amount of 2 wt. % and zinc in an amount of 5 wt. %, a small drop of conversion and significant increase of selectivity to 1,3-butadiene are observed when adding acetaldehyde in an amount of 10 vol. % to the ethanol feed, resulting in a high 1,3-butadiene yield.

Example 14 is a production process of 1,3-butadiene according to the invention. The catalyst system comprises a first stage catalyst of ZnOₓ—TaOₓ/SiO₂ and a second stage catalyst of TaOₓ/SiO₂. It performs best of all the examples at the chosen reaction conditions of 350° C. and a WHSV of 0.54 h⁻¹, even in the absence of acetaldehyde in the first stage feed. This is due to the fact that the first stage catalyst produces both acetaldehyde and 1,3-butadiene. Unused first stage (ethanol) feed and acetaldehyde produced in the first stage are converted to 1,3-butadiene in the second stage, increasing conversion and selectivity to (and thus yield of) 1,3-butadiene.

1. A process for the production of 1,3-butadiene, the process comprising the following stages
i) a first stage contacting of a first stage feed comprising ethanol with a first stage catalyst,
wherein the first stage catalyst comprises element MA1 and element MB1,
MA1 is selected from the group consisting of zinc, copper, silver, gold, chromium, cerium, magnesium, platinum, palladium, cadmium, iron, manganese, ruthenium, cobalt, and nickel, and
MB1 is selected from the group consisting of tantalum, zirconium, niobium, hafnium, titanium, and tin,
to produce a first stage effluent comprising acetaldehyde and 1,3-butadiene,
ii) a second stage contacting of a second stage feed with a second stage catalyst,
the second stage feed comprising at least part of the first stage effluent, and the second stage feed comprising ethanol and acetaldehyde,
wherein the second stage catalyst comprises element MB2, and
MB2 is selected from the group consisting of tantalum, zirconium, niobium, hafnium, titanium, and tin,
to produce a second stage effluent comprising 1,3-butadiene.

2. The process according to claim 1, wherein the first and second stage catalyst are packed in contact with one another in a single packing without any inert filling between the two stages.

3. The process according to claim 1, wherein the first and the second stage catalysts are separated by an inert filling in a single packing.

4. The process according to claim 1, wherein the first and the second stage catalysts are located in separate reactors that are connected with one another in series.

5. The process according to any of the preceding claims, wherein MA1 is selected from the group consisting of zinc, copper, silver, chromium, magnesium, and nickel, preferably wherein MA1 is zinc.

6. The process according to any of the preceding claims, wherein MA1 is present in an oxide form, preferably wherein the first stage catalyst comprises zinc oxide.

7. The process according to any of the preceding claims, wherein MB1 is selected from the group consisting of tantalum, zirconium, niobium, hafnium, preferably wherein MB1 is tantalum.

8. The process according to any of the preceding claims, wherein MB1 is present in an oxide form, preferably wherein the first stage catalyst comprises tantalum oxide.

9. The process according to any of the preceding claims, wherein the first stage catalyst comprises, based on the total weight of the first stage catalyst,
a) zinc oxide in an amount of from 0.05 to 18 wt. %, preferably from 0.05 to 5 wt. %, more preferably from 0.05 to 1 wt. %, more preferably from 0.05 to 0.2 wt. %, most preferably about 0.1 wt. %, calculated as ZnO, and/or
b) tantalum oxide in an amount of from 1 to 13 wt. %, preferably from 2 to 3 wt. %, more preferably about 2 wt. %, calculated as Ta₂O₅.

10. The process according to any of the preceding claims, wherein the first stage catalyst is a supported catalyst,
preferably wherein the support is selected from the group consisting of ordered and non-ordered porous silica supports, aluminium oxide supports, aluminosilicate supports, clays, other porous oxide supports, and mixtures thereof,
more preferably wherein the support is silica.

11. The process according to any of the preceding claims, wherein MB2 is selected from the group consisting of tantalum, zirconium, niobium, hafnium, preferably wherein MB2 is tantalum.

12. The process according to any of the preceding claims, wherein MB2 is present in an oxide form, preferably wherein the second stage catalyst comprises tantalum oxide.

13. The process according to claim 12, wherein the second stage catalyst comprises tantalum oxide in an amount of from 1 to 13 wt. %, preferably from 1 to 11 wt. %, preferably from 2 to 3 wt. %, more preferably about 2 wt. %, calculated as Ta₂O₅, based on the total weight of the second stage catalyst.

14. The process according to any of the preceding claims, wherein the second stage catalyst is a supported catalyst,
preferably wherein the support is selected from the group consisting of ordered and non-ordered porous silica supports, aluminium oxide supports, aluminosilicate supports, clays, other porous oxide supports and mixtures thereof,
more preferably wherein the support is silica.

15. The process according to any of the preceding claims, wherein both the first and the second stage catalysts are supported catalysts.

16. The process according to any of the preceding claims, wherein the first stage catalyst comprises zinc and tantalum in a molar ratio of from 0.01 to 1, preferably from 0.1 to 0.7, more preferably from 0.1 to 0.2.

17. The process according to any of the preceding claims, wherein the first stage feed additionally comprises acetaldehyde,
preferably wherein the acetaldehyde concentration is within a range of from 2 to 30 vol. %, more preferably from 5 to 20 vol. %, most preferably from 7 to 15 vol. %, each based on total volume of the first stage feed.

18. The process according to any of the preceding claims, wherein the first stage catalyst is produced or producible according to a method comprising:
a) impregnating a first stage support comprising silica with a first stage catalyst precursor comprising an MA1 compound and an MB1 compound;
b) drying the impregnated first stage support; and
c) calcining the dried impregnated first stage support.

19. The process according to any of the claims 1 to 17, wherein the first stage catalyst is produced or producible according to a method comprising:
a) impregnating a first stage support comprising silica with a first stage catalyst precursor comprising an MA1 compound or an MB1 compound;
b) drying the impregnated first stage support;
c) calcining the dried impregnated first stage support;
d) impregnating the calcined dried impregnated first stage support with a first stage catalyst precursor comprising the other of an MA1 compound and an MB1 compound;
e) drying the impregnated calcined dried impregnated first stage support; and
f) calcining the dried impregnated calcined dried impregnated first stage support.

20. Catalyst system for use in the production of 1,3-butadiene from ethanol comprising
i) a first stage catalyst comprising element MA1 and element MB1, wherein
MA1 is selected from the group consisting of zinc, copper, silver, gold, chromium, cerium, magnesium, platinum, palladium, cadmium, iron, manganese, ruthenium, cobalt, and nickel, and
MB1 is selected from the group consisting of tantalum, zirconium, niobium, hafnium, titanium, and tin, and
ii) a second stage catalyst comprising element MB2, wherein
MB2 is selected from the group consisting of tantalum, zirconium, niobium, hafnium, titanium, and tin.

21. The catalyst system according to claim 20, wherein the first and second stage catalyst are packed in contact with one another in a single packing without any inert filling between the two stages.

22. The catalyst system according to claim 20, wherein the first and the second stage catalyst are separated by an inert filling in a single packing.

23. The catalyst system according to claim 20, wherein the first and the second stage catalysts are located in separate reactors that are connected with one another in series.

24. The catalyst system according to any of the preceding claims, wherein the first stage catalyst comprises zinc and tantalum, and the second stage catalyst comprises tantalum.

25. The catalyst system according to any of the preceding claims, wherein the first stage catalyst comprises (each based on the total weight of the first stage catalyst)
zinc oxide, preferably in an amount of from 0.05 to 18 wt. %, more preferably from 0.05 to 5 wt. %, more preferably from 0.05 to 1 wt. %, more preferably from 0.05 to 0.2 wt. %, most preferably about 0.1 wt. %, calculated as ZnO, and/or
tantalum oxide, preferably in an amount of from 1 to 13 wt. %, more preferably from 2 to 3 wt. %, most preferably about 2 wt. %, calculated as Ta₂O₅.

26. Use of a catalyst system as defined in any one of the claims 20 to 25 for the production of 1,3-butadiene from a feed comprising ethanol,
preferably to decrease the required amount of acetaldehyde in the first stage feed or to dispense altogether with acetaldehyde in the first stage feed.

27. A plant comprising the catalyst system of any of claims 20 to 25.

28. The plant according to claim 27, wherein
the catalyst system is contained in one reactor,
or wherein the first stage catalyst and the second stage catalyst of the catalyst system are contained in separate reactors that are connected in series.
